(19) United States
(12) Patent Application Publication
(10) Pub. No.: US 2026/0015301 A1
(43) Pub. Date: Jan. 15, 2026

(54) SYSTEMS AND PROCESSES FOR THE PRODUCTION OF OLEFINIC PRODUCTS

(71) Applicant: SABIC GLOBAL TECHNOLOGIES B.V., BERGEN OP ZOOM (NL)

(72) Inventors: Abdulrahman Shahid SARANG, Bangalore (IN); Kenneth Francis LAWSON, Bangalore (IN); Ravichander NARAYANASWAMY, Bangalore (IN); Ahmad M. AL-SHEHRI, Bangalore (IN); Hatem Abdallah BELFADHEL, Bangalore (IN)

(73) Assignee: SABIC GLOBAL TECHNOLOGIES B.V., BERGEN OP ZOOM (NL)

(21) Appl. No.: 18/992,668

(22) PCT Filed: Jul. 6, 2023
(86) PCT No.: PCT/EP2023/068775

§ 371 Date: Jan. 9, 2025

(30) Foreign Application Priority Data
Jul. 9, 2022 (IN) 202241039485

(51) Int. Cl. C07C 4/04; B01J 19/24; C10G 9/36

(52) U.S. Cl. CPC C07C 4/04; B01J 19/245; C10G 9/36; C10G 2300/1081; C10G 2400/20

(57) ABSTRACT

Systems and processes for producing hydrocarbon products from a hydrocarbon feedstock are described. A system can a steam cracking unit capable of receiving ethane from an ethane source, ethane/propane from an ethane/propane source, propane/butane from a propane/butane dehydrogenation unit, or a combination thereof. Processes to produce hydrocarbon products are also described

### CROSS REFERENCE TO RELATED APPLICATIONS

None.

### BACKGROUND OF THE INVENTION

### A. Field of the Invention

The invention generally concerns systems and processes for producing hydrocarbon products from hydrocarbon feedstocks. A system can include a steam cracking unit capable of receiving ethane, optional propane, and/or a dehydrogenated propane/butane composition. The steam cracking unit is coupled to a feed preparation unit and an optional propane/butane dehydrogenation unit.

### B. Description of Related Art

Olefins (e.g., ethylene), are basic building blocks for a variety of commercially valuable polymers. Naturally occurring sources of olefins do not exist in commercial quantities. Therefore, polymer producers rely on methods for converting the more abundant lower alkanes into olefins. The method of choice for today's commercial scale producers is steam cracking, a highly endothermic process where steam-diluted alkanes are subjected briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shut-down for maintenance and coke removal.

Processes and systems to produce olefins from crude oil have been described. For example International Patent Application No. WO 2017/133975 to Ward et al., describes an integrated process to convert crude oil into petrochemical products that includes crude oil distillation, hydrocracking, and steam cracking to produce petroleum products. Some processes use a Mixed Feed Cracker (MFC). A MFC can handle from light hydrocarbons such as ethane, propane, and butane through naphtha up to heavy liquid feedstocks such as gas oils and hydrocracker residues to produce gasoline and a majority of petrochemical industry products. A MFC process can be operated depending on the optimized downstream value chain between the oil or gas availability as feedstock and the market prices in high added value hydrocarbon products. However, mixed feed steam cracker suffer in that they do no convert all material to olefins. Optimal performance of cracking units occurs when fed with compatible feeds. In most steam cracking units, a dedicated furnace is usually required for optimal ethane cracking. This results in different types and sizes of furnaces. Steam cracking can result in the slow deposition of coke, a form of carbon, on the reactor walls. Decoking requires the furnace to be isolated from the process and then a flow of steam or a steam/air mixture is passed through the furnace coils. This converts the hard solid carbon layer to carbon monoxide and carbon dioxide. Once this reaction is complete, the furnace is returned to service. Due to the amount of coking that occurs in the MRC when ethane is cracked, the MRC generally requires at least two compatible furnaces for the cracking of ethane, which can compromise the size of the furnaces in the MRC and make the process less efficient and more cost intensive.

Systems and processes to produce hydrocarbon products have been described. For example, U.S. Pat. No. 11,180,706 to A1-Sayed et al. describes a configuration for olefins production. The processes progressively separate a crude oil into light and heavy fractions, which can be upgraded using fixed bed hydroconversion unit, a fluidized catalytic conversion unit, or a residue hydrocracking unit. The upgraded fluids can be fed to a steam cracking unit for production of olefins. This process suffers from less than optimal performance, which can increase capital cost of the overall process and lower profitability.

Overall, while the technologies of producing petrochemicals (e.g., olefins) exist, they can be energy inefficient and expensive.

### SUMMARY OF THE INVENTION

A discovery has been made that provides a solution to at least one of the problems associated with production of petrochemicals from crude oil. In one aspect, the discovery can include a system that provides an ethane/propane source, a butene/propene source, or both, coupled to a steam cracking unit. The butene/propene source can be a butane/propane dehydrogenation unit positioned downstream of the steam cracking unit. This set-up can provided higher ethylene selectivity/conversion by providing hydrogen-rich feeds like ethane, ethene, propane, propene, butane, or butenes, or mixtures thereof, to the steam cracking unit. Also, this setup up allows an increase in the amount of other hydrocarbons, for example, naphtha, gas oil and the like, that can be fed to the steam cracking unit or made into other petroleum products (e.g., fuels, lubricants, and the like). In some aspects, this set-up can also allow for overall conversion of crude oil to petrochemical products to be at least 50%, preferably 65%, more preferable at least 70%.

In one aspect of the present invention, systems and processes to produce hydrocarbon products such as olefins are described. A system for producing hydrocarbon products can include a feed preparation unit, a steam cracking unit, and an ethane and/or propane feed stream conduit. The feed preparation unit can be capable of producing, from crude oil, a hydrocarbon feed stream comprising liquid petroleum gas (LPG), hydrocarbons, or a mixture thereof, and a fuels/pitch stream. The feed preparation unit can also be referred to as a crude oil processing unit, which can be coupled to the steam cracker unit such that the hydrocarbon feed stream can be capable of being delivered to the steam cracker unit. The steam cracking unit is capable of cracking hydrocarbons, preferably ethane and/or propane, to produce ethylene and/or propylene. The ethane and/or propane feed stream conduit can be separate from the hydrocarbon feed stream and can be capable of delivering ethane and/or propane to the steam cracker unit. In another aspect, the system can also include a propane/butane dehydrogenation unit coupled to the steam cracking unit. The propane/butane dehydrogenation unit can be capable of providing propene and butenes to the steam cracking unit. A recycle propane/butane conduit can be coupled to the steam cracking unit and the propane/butane dehydrogenation unit. Propane, butane, or both, can be transferred to the propane/butane dehydrogenation unit from the steam cracking unit via the recycle propane/butane conduit. A propane/butane conduit can provide additional propane, butane, or a mixture thereof to the propane/butane dehydrogenation unit. In some aspects, a feed preparation unit is coupled to the propane/butane dehydrogenation unit. The feed preparation unit can provide propane, butane, or a mixture thereof to the propane/butane dehydrogenation unit.

In another aspect of the invention, processes to produce olefins are described. A process can include subjecting a hydrocarbon feed stream that includes a mixture of liquid petroleum gas (LPG) and/or higher boiling hydrocarbons and a second gaseous feed stream that includes ethane, propane, or a mixture thereof to steam cracking conditions sufficient to produce olefinic products. The hydrocarbon feed stream can include liquid petroleum gas, hydrocarbons, or a combination thereof. In some aspects, the second gaseous feed stream can include primarily ethane, butane, or a mixture, thereof feed stream (e.g., at least 50 wt. % ethane, at least 75 wt. % ethane, at least 99 wt. % ethane, at least 50 wt. % butane, at least 75 wt. % butane, at least 99 wt. % butane, or at least 50 wt. % of a combination of ethane and butane, at least 75 wt. % of a combination of ethane and butane, or at least 99 wt. % of a combination of ethane and butane) obtained from a source different than the LPG and/or the higher boiling hydrocarbons. Steam cracking conditions can include a temperature of 600° C. to 1000° C. and a pressure of 0.1 MPa to 0.5 MPa. In some aspects, the steam cracking conditions can produce a first (C3/C4) hydrocarbon stream that can include propane, butane, or a combination thereof. The first (C3/C4) hydrocarbon stream, a second (C3/C4) hydrocarbon stream that includes propane, butane, or a combination thereof, or a combination of the first (C3/C4) hydrocarbon stream and the second (C3/C4) hydrocarbon stream can be subjected to conditions suitable to produce a dehydrogenated C3/C4 composition (e.g., propylene, isobutylene, butadiene, butene, or a combination thereof). In some aspects, a crude oil feed can be subject to conditions suitable to produce the hydrocarbon feed stream. Such processing can include producing a third (C3/C4) hydrocarbon stream from the crude oil feed that includes propane, butane, or a combination thereof. This third (C3/C4) hydrocarbon stream can be combined with the second (C3/C4) hydrocarbon stream and subjected to conditions suitable to produce the dehydrogenated propane/butane composition. The crude oil conditions can also produce fuels and/or pitch from the crude oil, which can be separated to produce a fuels composition and a pitch composition. The fuels composition can include diesel fuel, kerosene, gas oil, or a blend thereof. Olefinic products produced from steam cracking can include ethylene, propylene, or a mixture thereof.

In another aspect of the present invention there is disclosed a system and process for producing hydrocarbons such as ethylene and/or propylene. The system can use at least two, three, four, or more steam cracking units that can be positioned in parallel or series, or a combination thereof, with each other. An advantage of this set up is that conversion of crude oil into petrochemical products such as ethylene and/or propylene can be at least 50%, preferably 65%, more preferable at least 70%, 80%, 90%, or more. A system can include a feed preparation unit capable of producing, from crude oil, a hydrocarbon feed stream comprising liquid petroleum gas (LPG), hydrocarbons, or a mixture thereof, and a fuels/pitch stream, a first steam cracking unit positioned downstream and coupled to the feed preparation unit and capable of cracking hydrocarbons from the hydrocarbon feed stream, preferably ethane and/or propane, to produce a C2/C3 product stream comprising ethylene and/or propylene, and a second steam cracking unit capable of cracking hydrocarbons from the hydrocarbon feed stream and/or hydrocarbons from the C2/C3 product stream. The second steam cracking unit can be positioned downstream and coupled to the feed preparation unit or can be positioned downstream and coupled to the first steam cracking unit. In one aspect, the second steam cracking unit can be positioned downstream and coupled to the feed preparation unit, wherein the first steam cracking unit is capable of cracking a first portion of the hydrocarbon feed stream, and the second steam cracking unit is capable of cracking a second portion of the hydrocarbon feed stream. In another aspect, the second steam cracking unit can be positioned downstream and coupled to the first steam cracking unit, wherein the second steam cracking unit is capable of cracking hydrocarbons from the C2/C3 product stream produced by the first steam cracking unit. Also disclosed are methods of producing ethylene and/or propylene with the system having two or more steam cracking units.

Coupling can be direct fluid communication with the coupled units or indirect fluid communication with the coupled units.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The term “C# hydrocarbons”, wherein “#” is a positive integer, is meant to describe all hydrocarbons having # carbon atoms. Moreover, the term “C#+ hydrocarbons” is meant to describe all hydrocarbon molecules having # or more carbon atoms. Accordingly, the term “C2+hydrocarbons” is meant to describe a mixture of hydrocarbons having 2 or more carbon atoms. The term “C2+alkanes” accordingly relates to alkanes having 2 or more carbon atoms.

“Cracking” refers to a process involving decomposition and molecular recombination of organic compounds to produce a greater number of molecules than were initially present. In cracking, a series of reactions take place accompanied by a transfer of hydrogen atoms between molecules. For example, naphtha may undergo a thermal cracking reaction to form ethene and hydrogen.

“Hydrocarbons” are generally defined as molecules formed primarily by carbon and hydrogen atoms. Hydrocarbons may also include other elements such as, but not limited to, halogens, metallic elements, nitrogen, oxygen, and/or sulfur. Hydrocarbon fluids may include, entrain, or be entrained in non-hydrocarbon fluids such as hydrogen, nitrogen, carbon monoxide, carbon dioxide, hydrogen sulfide, water, and/or ammonia.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The systems and processes of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the systems and methods of the present invention are their abilities to produce petrochemical products (e.g., olefins such as ethylene or propylene) in a cost and energy efficient manner from crude oil.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 illustrates an embodiment of a system to produce olefin products that includes a steam cracking unit coupled to ethane/propane feed source.

FIG. 2 illustrates an embodiment of another system to produce olefinic products that includes a propane/butane dehydrogenation unit coupled to a steam cracking unit.

FIG. 3 illustrates an embodiment of another system to produce olefinic products that includes a feed preparation unit coupled to a propane/butane dehydrogenation unit coupled to a steam cracking unit.

FIGS. 4A and 4B illustrate embodiments of another system to produce olefinic products that includes two steam cracking units, where one of the steam cracking units is positioned parallel to the other steam cracking unit (FIG. 4A), and where one of the steam cracking units is positioned in series with the other steam cracking unit (FIG. 4B).

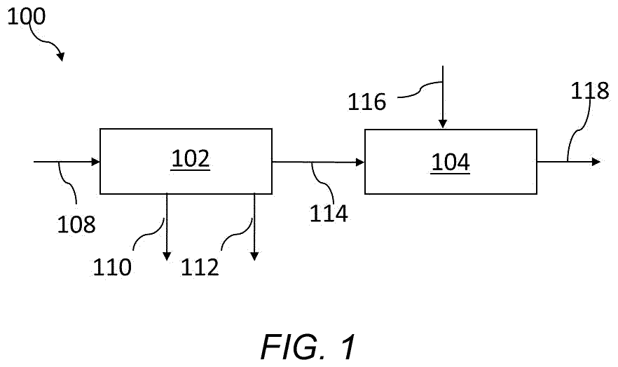

While the invention is susceptible to various modifications and alternative forms, specific embodiments thereof are shown by way of example in the drawings. The drawings may not be to scale. Still further, the schematics illustrated in FIGS. 1-4 can be combined with one another, which can be used, for example, to create a more robust process of producing a variety of petrochemical products from crude oil.

### DETAILED DESCRIPTION OF THE INVENTION

A discovery has been made that provides a solution to at least one of the problems associated with producing petrochemicals from crude oil. In one aspect of the invention, a hydrocarbon feed obtained from crude oil and a separate alkane feed (e.g., a feed comprising primarily (i.e., 50 wt. % or more, 60 wt. % or more, 70 wt. % or more, 80 wt. % or more, 90 wt. % or more, or 99 wt. % or more, ethane and/or butane) can be fed to a steam cracking unit to produce petroleum products. An advantage of this set-up is that higher hydrogen feeds are fed to the steam cracking unit, which can increase the amount of ethylene and propylene produced. In addition, this allows more of the processing of the crude oil to be directed to the production of fuels. These and other non-limiting aspects of the present invention are discussed in further detail in the following sections with reference to the Figures.

Referring to FIG. 1, system 100 for producing olefin products is described. System 100 can include a feed preparation unit 102 and a steam cracking unit 104. Crude oil 108 enters feed preparation unit 102. Crude oil can be the petroleum extracted from geologic formations in its unrefined form. The term crude oil can also include petroleum that has been subjected to water-oil separations and/or gas-oil separation and/or desalting and/or stabilization. Non-limiting examples of crude oil include Arabian Heavy, Arabian Light, other Gulf crudes, Brent, North Sea crudes, North and West African crudes, Indonesian, Chinese crudes, West Texas crude, and mixtures thereof, but also shale oil, tar sands, gas condensates and bio-based oils. The crude oil used as feed to the process of the present invention preferably is conventional petroleum having an API gravity of more than 20° API as measured by the ASTM D287 standard. In one aspect, the crude oil used in the process of the present invention is a light crude oil having an API gravity of more than 30° API. In another aspect, the crude oil used in the process of the present invention can include Arabian Light Crude Oil. Arabian Light Crude Oil typically has an API gravity of between 32-36° API and a sulfur content of between 1.5-4.5 wt. %. The crude oil can be separated into different crude oil fractions based on a difference in boiling point. For example, the crude oil can be distilled using a fractionating column, or a combination of more than one fractionation column, that is used to separate crude oil into fractions by fractional distillation. The resulting crude oil can be processed in an atmospheric distillation unit to separate gas oil and lighter fractions from higher boiling components to produce a mixed hydrocarbon feed 110. The hydrocarbon feed can include hydrocarbons having a boiling point above 560° C. Non-limiting examples of various distillate hydrocarbon feeds having a boiling point above 560° C. include vacuum gas oil, middle distillate, naphtha, kerosene, liquid petroleum gas. The distillate hydrocarbons can be converted into fuels and/or additional feed for the steam cracking unit. Pitch 112 can also be produced from feed preparation unit 102. Fuels stream 110 can exit feed preparation unit 102 and be sold, transported, or further processed. Pitch stream 112 can exit feed preparation unit 102 and be further processed.

Hydrocarbon feed 114 can exit feed preparation unit 102 and enter steam cracking unit 104. Hydrocarbon feed can include ethane, propane, butanes, naphtha, distillates, or any combination thereof. Second gaseous hydrocarbon stream 116 can enter steam cracking unit 104. Second gaseous hydrocarbon stream 116 can include, ethane, propane, or a mixture thereof. Second gaseous hydrocarbon stream 116 can include 50 wt. % or more, 60 wt. % or more, 70 wt. % or more, 80 wt. % or more, 90 wt. % or more, or 99 wt. % or more, ethane or butane or a combination of ethane and butane. In steam cracking unit 104, the hydrocarbon feed 114 and the second gaseous hydrocarbon feed 116 can be subjected to steam cracking at a temperature of 600° C. to 770° C. (e.g., 600° C., 625° C., 650° C., 675° C., 700° C., 725° C., 950° C., 770° C., or any value or range there between) and/or a pressure of 0.2 MPa to 0.3 MPa (e.g., 0.2 MPa, 0.21 MPa, 0.22 MPa, 0.23 MPa, 0.24 MPa, 0.25 MPa, 0.26 MPa, 0.27 MPa, 0.28 MPa, 0.30 MPa, or any value or range there between). In a steam cracking process, the saturated hydrocarbons are broken down into smaller, often unsaturated, hydrocarbons such as ethylene and propylene by diluting the mixed hydrocarbon feed with steam and heating the mixture in a furnace in the absence presence of oxygen. The steam cracking reaction can have a residence times of 50-1000 milliseconds. Mixed steam cracking unit can have a fractionation unit (not shown) or a gas fractionation unit (not shown) capable of the olefin product stream from other products (e.g., butane, propane, and the like). Such fractionation units are well known in the art. Olefin product stream 118 can exit mixed feed steam cracking unit 104 and be stored, transported or used in other processing units.

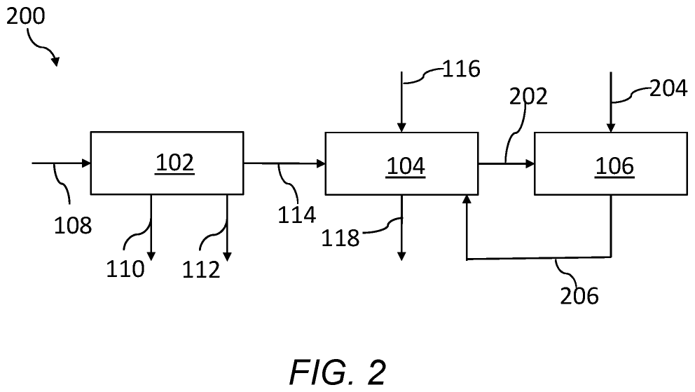

Referring to FIG. 2, additional hydrogen rich alkenes (e.g., propenes and/or butenes) can be provided to steam cracking unit 104 from propane/butane dehydrogenation unit 106. Propane/butane dehydrogenation unit 106 can receive first C3/C4 alkane stream 202 from steam cracking unit 104, second C3/C4 alkane stream 204 from a propane and/or butane source, or a combination thereof. The C3/C4 alkane streams can include propane, butane, or a mixture thereof. The ratio of C3 to C4 alkanes in the C3/C4 streams can be from 0.1 to 99.9 to 99.9 to 0.1 and can vary depending on the requirements of the steam cracking unit, the propane/butane dehydrogenation unit or both. The amount of propane and/or butane in the C3/C4 streams can be at least 50 wt. % or more, 60 wt. % or more, 70 wt. % or more, 80 wt. % or more, 90 wt. % or more, or 99 wt. % or more, propane and/or butane). In some embodiments, the C3/C4 alkanes can be provided to propane/butane dehydrogenation unit 106 in separate streams (e.g., a C3 stream and a C4 stream). In propane/butane dehydrogenation unit, propane, butane, or a mixture thereof can be dehydrogenated to propene, butenes, or a mixture thereof (e.g., propylene, isobutylene, butene, butadiene, and the like). Dehydrogenated propene/butenes (C3/C4 alkenes) stream 206 can exit propane/butane dehydrogenation unit and enter steam cracking unit 104. Dehydrogenation conditions can include 500° C. to 1000° C., a pressure in a range of 0 to 1379 kPa, and a space velocity of 0.1 to 10 hr−1 weight hourly space velocity (WHSV). A portion of the dehydrogenated propene/butenes (C3/C4 alkenes) stream 206 can be provided to other processing units, stored, or transported. In some embodiments, C3/C4 alkane stream 202 can be considered a recycle stream between the steam cracking unit and the propane/butane dehydrogenation unit as sufficient C3/C4 alkanes may not be produced prior to providing the dehydrogenated propene/butenes to the steam cracking unit.

In steam cracking unit 104, second gaseous hydrocarbon stream 116 can include mostly ethane (e.g., at least 50 wt. %, 60 wt. %, 70 wt. %, 80 wt. %, 90 wt. %, 91 wt. %, 92 wt. %, 93 wt. %, 94 wt. %, 95 wt. %, 96 wt. %, 97 wt. %, 98 wt. %, 99 wt. % or 100 wt. % ethane or any range or value there between). In steam cracking unit 106, gaseous ethane, optional propane, dehydrogenated propane/butane composition (e.g., propene, and butenes) can be diluted with steam and heated to a temperature of 775° C. to 860° C. (e.g., 775° C., 800° C., 825° C., 850° C., 860° C., or any value or range there between) and/or a pressure of 0.2 MPa to 0.3 MPa (e.g., 0.2 MPa, 0.21 MPa, 0.22 MPa, 0.23 MPa, 0.24 MPa, 0.25 MPa, 0.26 MPa, 0.27 MPa, 0.28 MPa, 0.30 MPa, or any value or range there between) in one or more furnaces to produce olefins and hydrocarbon products. Steam cracking unit 104 can include one or more separation devices that can separate, propane, butane or a mixture thereof from the olefinic products and other hydrocarbon products. The separated propane and/or butane can form second propane/butane product stream. Olefin product stream 118 can exit mixed feed steam cracking unit 104 and be stored, transported or used in other processing units. In some embodiments, olefinic product stream 118 can have an increased amount of ethylene and or propylene as compared to steam cracking of hydrocarbon feed in the absence of additional ethane, propane, and/or butane.

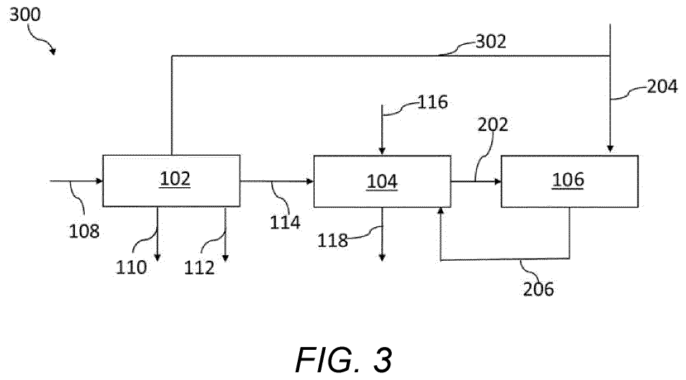

Referring to FIG. 3, the system of FIG. 2 is described with the addition of propane and/or butane being provided from the feed preparation unit to the propane/butane dehydrogenation unit. In system 3, a third C3/C4 hydrocarbon stream 302 can be produced from the feed preparation unit by separating propane and/or butane from distillate, gas oil, vacuum gas oil, naphtha, or resid. Third C3/C4 alkane stream 302 can exit feed preparation unit 102 and be combined with first C3/C4 alkane hydrocarbon stream 202 (shown), be provided direction to propane/butane dehydrogenation unit (not shown), or a combination thereof. In some aspects, C3/C4 alkane stream 302 is provided to the steam cracking unit 104 (not shown).

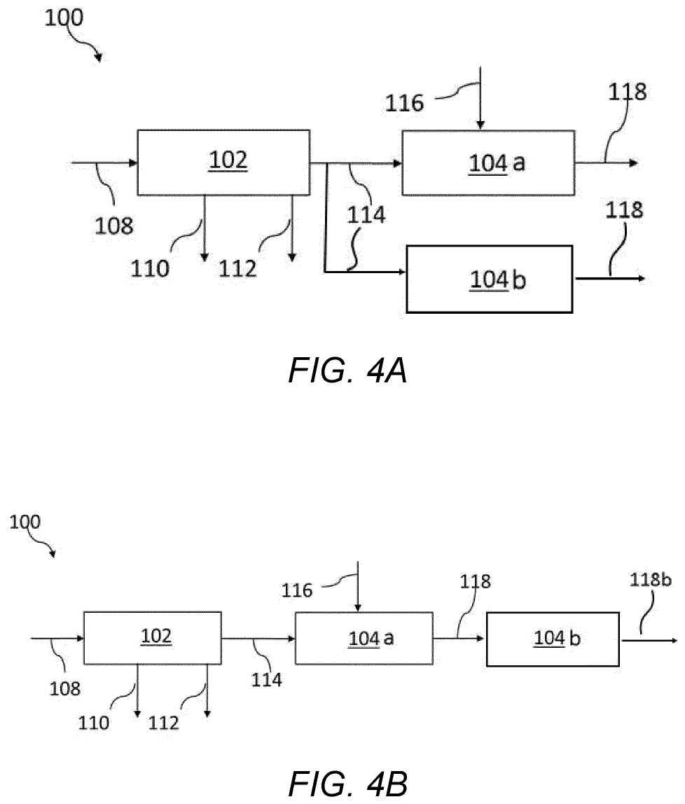

Referring to FIG. 4A, this is the system of FIG. 1, with an additional steam cracking unit. In particular, a first steam cracking unit 104a and a second steam cracking unit 114b are positioned in parallel to one another. A first portion of hydrocarbon feed 114 can be processed by the first steam cracking unit 104a, and a second portion of the hydrocarbon feed 114 can be processed by the second steam cracking unit 104b. This can be advantageous in producing additional olefinic product streams 118. Also, second hydrocarbon gas stream 116 can be removed from the system in FIG. 4A (not shown). Alternatively, another hydrocarbon gas stream 116 can be sent to the second steam cracking unit 104b (not shown). The system in FIG. 4A can include three, four, or more steam cracking units that can be positioned in parallel or series, or a combination thereof, with each other.

Referring to FIG. 4B, this is the system of FIG. 1, with an additional steam cracking unit. In particular, a first steam cracking unit 104a and a second steam cracking unit 104b are positioned in series to one another. The second steam cracking unit 104b is positioned downstream from the first steam cracking unit 104a. This set up allows for any uncracked hydrocarbons in product stream 118 to be further cracked in the second steam cracking unit 104b. The product stream 118b can have a higher content of olefinic products when compared with product stream 118, as uncracked hydrocarbons in 118 are subjected to another round of cracking in second steam cracking unit 104 (b). This can be advantageous in producing additional olefinic products (e.g., ethylene and/or propylene). Also, second hydrocarbon gas stream 116 can be removed from the system in FIG. 4B (not shown). Alternatively, another hydrocarbon gas stream 116 can be sent to the second steam cracking unit 104b (not shown). The system in FIG. 4B can include three, four, or more steam cracking units that can be positioned in parallel or series, or a combination thereof, with each other.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A process for the production of olefins, the process comprising subjecting a hydrocarbon feed stream comprising liquid petroleum gas (LPG), hydrocarbons, or a mixture thereof, and a second gaseous feed stream comprising ethane, propane, or a mixture thereof, to steam cracking conditions sufficient to produce olefinic products.

2. The process of claim 1, wherein the hydrocarbons comprise naphtha, gas oil, distillate, or a blend thereof.

3. The process of claim 1, wherein the second gaseous feed stream is obtained from a source different than the hydrocarbon feed.

4. The process of claim 1, wherein the steam cracking conditions comprise a temperature of 600° C. to 1000° C. and a pressure of 0.1 MPa to 0.5 MPa.

5. The process of claim 1, wherein crude oil is subject to conditions to produce the hydrocarbon feed.

6. The process of claim 5, wherein the crude oil is subject to conditions to produce fuels and/or pitch.

7. The process of claim 6, wherein the fuels comprises diesel fuel, kerosene, gas oil, or a blend thereof.

8. The process of claim 1, wherein the olefinic products produced from steam cracking comprise ethylene, propylene, C4 hydrocarbons, or a mixture thereof.

9. The process of claim 1, wherein the second gaseous feed stream comprises at least 50 wt. % ethane or propane, or a mixture thereof, preferably the second gaseous feed stream comprises at least 50 wt. % ethane.

10. The process of claim 9, wherein the steam cracking conditions produces a first (C3/C4) hydrocarbon stream comprising propane, butane, or a combination thereof.

11. The process of claim 10, wherein the first (C3/C4) hydrocarbon stream, a second (C3/C4) hydrocarbon stream comprising propane, butane, or a combination thereof, or a combination of the first (C3/C4) hydrocarbon stream and the second (C3/C4) hydrocarbon stream are subjected to conditions suitable to dehydrogenate the propane and/or butane, and wherein the dehydrogenated propane and/or butane is subjected to the steam cracking conditions.

12. The process of claim 1, further comprising subjecting a crude oil feed to conditions suitable to produce the hydrocarbon feed stream and a third C3/C4 hydrocarbon feed stream, preferably wherein the third C3/C4 feed stream is subjected to conditions to dehydrogenate the propane and/or butane, and wherein the dehydrogenated propane and/or butane is subjected to the steam cracking conditions.

13. The process of claim 1, further comprising subjecting the produced olefinic products to a second steam cracking step in a separate steam cracking unit to increase the amount of olefinic products produced.

14. The process of claim 1, further comprising subjecting a first portion of the hydrocarbon feed stream to the steam cracking conditions and subjecting a second portion of the hydrocarbon feed stream to a second steam cracking step, wherein the first portion is cracked in a first steam cracking unit and the second portion is cracked in a second steam cracking unit.

15. A system for producing hydrocarbon products, the system comprising:
a feed preparation unit capable of producing, from crude oil, a hydrocarbon feed stream comprising liquid petroleum gas (LPG), hydrocarbons, or a mixture thereof, and a fuels/pitch stream;
a steam cracking unit capable of cracking hydrocarbons, preferably ethane and/or propane, to produce ethylene and/or propylene, wherein the feed preparation unit is coupled to the steam cracker unit such that the hydrocarbon feed stream is capable of being delivered to the steam cracker unit; and
an ethane and/or propane feed stream conduit that is separate from the hydrocarbon feed stream and is capable of delivering ethane and/or propane to the steam cracker unit.

16. The system of claim 15, further comprising a propane/butane dehydrogenation unit coupled to the steam cracking unit, the propane/butane dehydrogenation unit capable of providing propene and butenes to the steam cracking unit, and/or
further comprising a recycle propane/butane conduit coupled to the steam cracking unit and the propane/butane dehydrogenation unit, the recycle conduit capable of providing propane, butane, or both to the propane/butane dehydrogenation unit from the steam cracking unit, and/or
further comprising a propane/butane conduit coupled to the propane/butane dehydrogenation unit and capable of providing propane, butane or a mixture thereof the propane/butane dehydrogenation unit, and/or
wherein the feed preparation unit is also capable of producing, from the crude oil, propane, butane or a combination thereof, and wherein the propane, butane, or the combination thereof is capable of being provided to the propane/butane dehydrogenation unit.

17. The system of claim 1, further comprising at least a second steam cracking unit capable of cracking hydrocarbons, preferably ethane and/or propane, to produce ethylene and/or propylene.

18. The system of claim 17, wherein the feed preparation unit is coupled to the second steam cracking unit such that at least a portion of the hydrocarbon feed stream is capable of being delivered to the steam cracking unit and a second portion of the hydrocarbon feed stream is capable of being delivered to the second steam cracking unit.

19. The system of claim 18, wherein the second steam cracking unit is positioned downstream from the steam cracking unit, and wherein the second steam cracking unit is coupled to the steam cracking unit and is capable of receiving C2/C3 products from the steam cracking unit.

20. A system for producing hydrocarbon products, the system comprising:
a feed preparation unit capable of producing, from crude oil, a hydrocarbon feed stream comprising liquid petroleum gas (LPG), hydrocarbons, or a mixture thereof, and a fuels/pitch stream;
a first steam cracking unit positioned downstream and coupled to the feed preparation unit and capable of cracking hydrocarbons from the hydrocarbon feed stream, preferably ethane and/or propane, to produce a C2/C3 product stream comprising ethylene and/or propylene; and
a second steam cracking unit capable of cracking hydrocarbons from the hydrocarbon feed stream and/or hydrocarbons from the C2/C3 product stream, wherein the second steam cracking unit is positioned downstream and coupled to the feed preparation unit or is positioned downstream and coupled to the first steam cracking unit.

21. The system of claim 20, wherein the second steam cracking unit is positioned downstream and coupled to the feed preparation unit, wherein the first steam cracking unit is capable of cracking a first portion of the hydrocarbon feed stream, and the second steam cracking unit is capable of cracking a second portion of the hydrocarbon feed stream, and/or
wherein the second steam cracking unit is positioned downstream and coupled to the first steam cracking unit, wherein the second steam cracking unit is capable of cracking hydrocarbons from the C2/C3 product stream.

22. A method of producing ethylene and/or propylene, the method comprising using the system of claim 20.
